(19) United States
(12) Patent Application Publication
(10) Pub. No.: US 2026/0248484 A1
(43) Pub. Date: Aug. 27, 2026

(54) Cardiac Output Measuring Device and A Method of Measuring Cardiac Output

(71) Applicant: Aequenimitas Enterprises, LLC, Doylestown, PA (US)

(72) Inventor: James W. Hansen, Doylestown, PA (US)

(73) Assignee: Aequenimitas Enterprises, LLC, Doylestown, PA (US)

(21) Appl. No.: 19/060,043

(22) Filed: Feb. 21, 2025

(51) Int. Cl. A61B 8/12; A61B 8/00; A61B 8/06; A61B 8/08; A61B 90/00; G16H 50/30

(52) U.S. Cl. CPC A61B 8/12; A61B 8/065; A61B 8/0883; A61B 8/445; A61B 8/4477; A61B 8/488; A61B 8/5223; A61B 90/06; G16H 50/30; A61B 2090/064

(57) ABSTRACT

A cardiac output measuring device includes a catheter, with an imaging sensor and a flow sensor on the catheter. The catheter has a distal end and a proximal end. The imaging sensor is positioned at the distal end and measures a cross-sectional area of a measurement location. The flow sensor is adjacent to the imaging sensor and measures a velocity of a fluid passing through the measurement location.

### FIELD OF THE INVENTION

The present invention relates to a measuring device and a method of measuring and, more particularly, to a cardiac output measuring device and a method of measuring cardiac output.

### BACKGROUND

Cardiac assessments of patients are commonly based on a measurement of cardiac output (“CO”). The CO is the amount of blood ejected from the left side of the heart per minute (measured in liters per minute or L/m), often calculated as stroke volume (“SV”, the amount of blood ejected from the left side of the heart per heart beat) multiplied by heart rate (“HR”, the number of heart beats per minute). Cardiac function can also be based on the cardiac index (“CI”), which is the CO normalized to a body surface area (BSA in square meters or m²) of the patient.

The CO is commonly measured using the Fick equation, thermodilution, or echocardiography; each of these methods, however, is based on a series of assumptions.

The Fick equation determines CO as a function of oxygen extraction across the peripheral arterial bed, which is evaluated with a right heart catheterization. Oxygen consumption is difficult to measure and, consequently, is often assumed in calculating CO according to this method. Oxygen consumption, however, can vary drastically between patients, which leads to inaccuracies in calculating CO. Further, left heart catheterizations do not have the capability to measure CO, the right heart catheterization is an invasive procedure requiring additional access points.

Cardiac output by thermodilution is performed through a two-lumen catheter; a proximal port is positioned in the right atrium and the distal tip is positioned in the pulmonary artery. A thermistor on the distal end measures a temperature of the blood. Room temperature saline is injected into the right atrium. The CO is derived based on the temperature drop at the thermistor. This method becomes increasingly inaccurate as CO drops to low levels and with significant valve disease.

Echocardiography can be used to estimate CO by measuring the velocity (centimeters per second or cm/s) of blood flow through the left ventricular outflow tract (“LVOT”), just below the aortic valve, and integrating the velocity (integration of cm/s yields a unit of cm). Multiplying the integrated velocity (cm) with a cross-sectional area (cm²) of the LVOT, assumed to be a circle, gives a stroke volume (cm³ which is equivalent to milliliters or mL) that can be used to calculate CO and CI. Assuming the cross-sectional area of the LVOT as a circle, however, leads to inaccuracies in calculation, as the LVOT is commonly an oval shape.

The assumptions in the current methods of calculating CO and CI lead to inaccuracy in the measurements and unreliable data for treatment.

### SUMMARY

A cardiac output measuring device includes a catheter, with an imaging sensor and a flow sensor on the catheter. The catheter has a distal end and a proximal end. The imaging sensor is positioned at the distal end and measures a cross-sectional area of a measurement location. The flow sensor is adjacent to the imaging sensor and measures a velocity of a fluid passing through the measurement location.

### BRIEF DESCRIPTION OF THE DRAWINGS

The invention will now be described by way of example with reference to the accompanying figures, of which:

FIG. 1 is a plan view of a cardiac output measuring device according to an exemplary embodiment;

FIG. 2A is an enlarged schematic sectional view of portion A of FIG. 1;

FIG. 2B is an enlarged schematic view of portion B of FIG. 1;

FIG. 2C is an enlarged schematic view of portion C of FIG. 1;

FIG. 3 is a schematic sectional view of a heart with the cardiac output measuring device of FIG. 1 positioned therein in a left ventricular outflow tract;

FIG. 4 is schematic view of a cardiac output measuring system according to an exemplary embodiment; and

FIG. 5 is a flowchart of a method of measuring a cardiac output according to an exemplary embodiment.

### DETAILED DESCRIPTION OF THE EMBODIMENT(S)

Exemplary embodiments of the present invention will be described hereinafter in detail with reference to the attached drawings, wherein like reference numerals refer to like elements. The present invention may, however, be embodied in many different forms and should not be construed as being limited to the embodiments set forth herein; rather, these embodiments are provided so that the present disclosure will convey the concept of the invention to those skilled in the art.

Throughout the drawings, only one, multiple, or all of a plurality of identical elements may be labeled in a figure for clarity of the drawings, but the detailed description of the element herein applies equally to each of the identically appearing elements in the figure if all elements are not labeled. A relative thickness, length, or width of the elements described in more detail below appear in the figures. The relative thickness, length, or width of the elements are merely illustrative and are not meant to be representative of a particular thickness, length, or width of any of the elements.

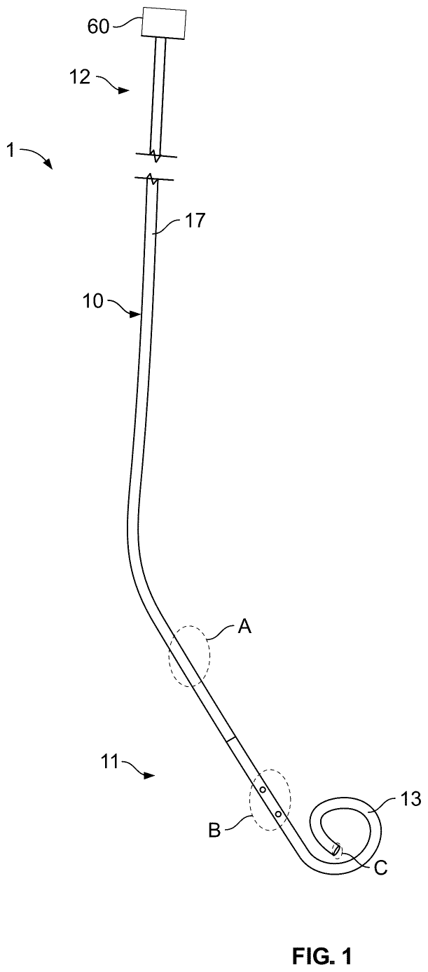

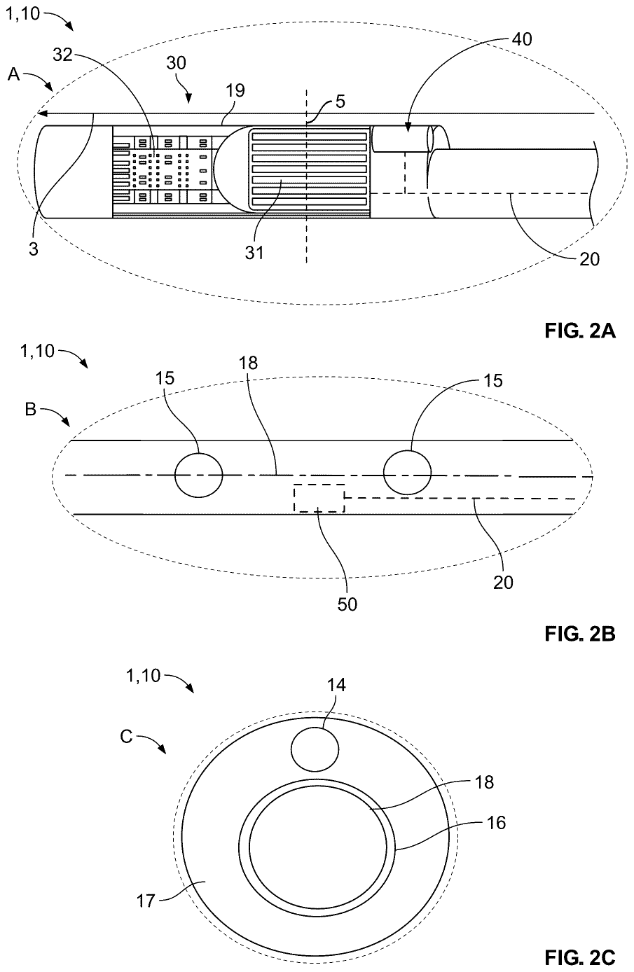

The cardiac output measuring device 1 according to an exemplary embodiment is now described with reference to FIGS. 1-2C.

As shown in FIG. 1, the cardiac output measuring device 1 includes a catheter 10. The catheter 10 extends from a proximal end 12 to a distal end 11 opposite the proximal end 12. The catheter 10 is a hollow tube that has a body 17 and an inner lumen 16, shown in FIG. 2C, extending between the proximal end 12 and the distal end 11. The inner lumen 16 is a passageway extending through the body 17. The body 17 of the catheter 10 may be formed of a polymer material. In other embodiments, the body 17 may be formed of any type of material used in catheters 10.

The distal end 11 is shown in detail in FIG. 1. As the distal end 11, the catheter 10 has a curved portion 13. In the shown embodiment, the curved portion 13 is curved in a uniform direction to nearly 360°; the catheter 10 is a pigtail catheter in the shown embodiment. In another embodiment, the curved portion 13 may not be curved or may be curved in any way and to any degree that allows the catheter 10 to function as described herein.

The catheter 10, as shown in FIGS. 2A and 2B, also includes a removable guidewire 18 and at least one electrical wire 20. The guidewire 18 and the at least one electrical wire 20 extend along a length of the catheter 10. The guidewire 18 is positioned in the inner lumen 16 and the at least one electrical wire 20 may be positioned anywhere within the body 17. The guidewire 18 of the present embodiment has a 0.035 inch diameter. In various other embodiments, the guidewire 18 may have any dimensions that allow the catheter 10 to function as described herein. The at least one electrical wire 20 may be any type of wire commonly used in catheter applications and capable of transmitting electrical signals and/or power.

The catheter 10 also includes at least one end hole 14 and at least one side hole 15. As shown in FIGS. 1 and 2C, the at least one end hole 14 is located on a portion of the body 17 at the end of the curved portion 13. As shown in FIGS. 1 and 2B, the at least one side hole 15 is adjacent to the curved portion 13. In another embodiment, the catheter 10 may have any number of side holes 15, which may be located anywhere on the distal end 11, including on the curved portion 13.

As shown in FIGS. 1 and 2A, the cardiac output measuring device 1 includes an imaging sensor 30. The imaging sensor 30 is positioned at the distal end 11. As shown in FIG. 2A, the imaging sensor 30 of the present embodiment has a transducer 31 and an imaging integrated circuit 32 connected to the transducer 31. The transducer 31 may be a solid state array transducer 31, such as a piezoelectric transducer 31. The imaging integrated circuit 32 may be any type of integrated circuit used in catheter applications and capable of performing the processing of the data gathered by the transducer 31 as described in detail below.

In an exemplary embodiment, the imaging sensor 30 is an ultrasound sensor. In a further exemplary embodiment, the imaging sensor 30 is a B-mode ultrasound sensor capable of obtaining a 360° image around the sensor 30. In various other embodiments, the imaging sensor 30 may be any type of sensor usable in a catheter that is capable of capturing an image suitable for determining a cross-sectional area data 33, as described below.

The cardiac output measuring device 1 includes a flow sensor 40, as shown in FIGS. 1 and 2A. As shown in FIG. 2A, the flow sensor 40 is positioned at the distal end 11 adjacent to the imaging sensor 30. The flow sensor 40, as shown in FIG. 2A, is oriented parallel to a plane 19 of the catheter 10 and perpendicular to the transducer 31. In another embodiment, the flow sensor 40 may be positioned anywhere on the catheter 10 and relative to the imaging sensor 30 that allows the cardiac output measuring device 1 to function as described herein. The flow sensor 40 of the present embodiment is a piezoelectric crystal that acts as a doppler sensor. In various other embodiments, the flow sensor 40 may be any type of sensor suitable for use in a catheter that is capable of measuring a velocity of a fluid 3 flowing adjacent to the flow sensor 40.

The cardiac output measuring device 1 also includes a pressure sensor 50, as shown in FIG. 2B. The pressure sensor 50 is in communication with the at least one end hole 14 and the at least one side hole 15. In the present embodiment, the pressure sensor 50 is a piezoelectric pressure sensor. In various other embodiments, the pressure sensor 50 may be any type of sensor suitable for use in a catheter that is capable of measuring a pressure of a fluid flowing through the at least one end hole 14 and/or the at least one side hole 15.

As shown in FIGS. 2A and 2B, each of the imaging sensor 30, the flow sensor 40, and the pressure sensor 50 is connected to one of the electrical wires 20 that extends through the catheter 10. The imaging sensor 30, the flow sensor 40, and the pressure sensor 50 can output measured values along the electrical wires 20.

In various other embodiments, the cardiac output measuring device 1 may include any number of additional sensors in the catheter 10 at the distal end 11, such as temperature sensors, oxygenation sensors, etc.

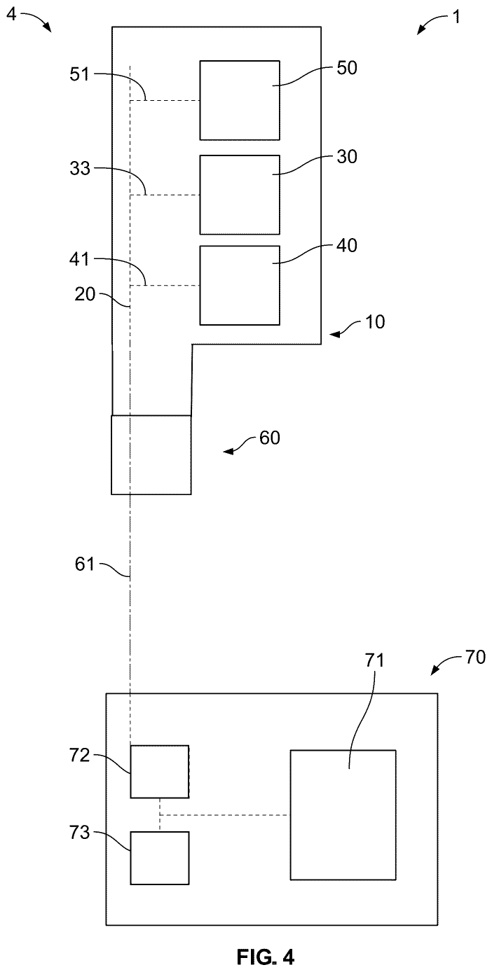

A cardiac output measuring system 4 according to an exemplary embodiment, as shown in FIG. 4, includes the cardiac output measuring device 1 described above, a connector 60, and a console 70. The connector 60, as shown in FIGS. 1 and 4, is positioned on the proximal end 12 of the catheter 10 and may be any type of electrical connector that can connect with the console 70. The console 70, as shown in FIG. 4, has a display 71. The display 71 displays an image and/or other data. As shown in FIG. 4, the console 70 also has a processor 72 and a memory 73 connected to the processor 72 with a plurality of algorithms stored thereon. The memory 73 is a non-transitory computer readable medium. Any functions that the console 70 is described as performing herein are executed by the processor 72 according to the algorithms stored on the memory 73. For example, the processor 72 is connected to the display 71 and, as described in detail below, controls the elements that appear on the display 71 and performs the calculations that appear on the display 71.

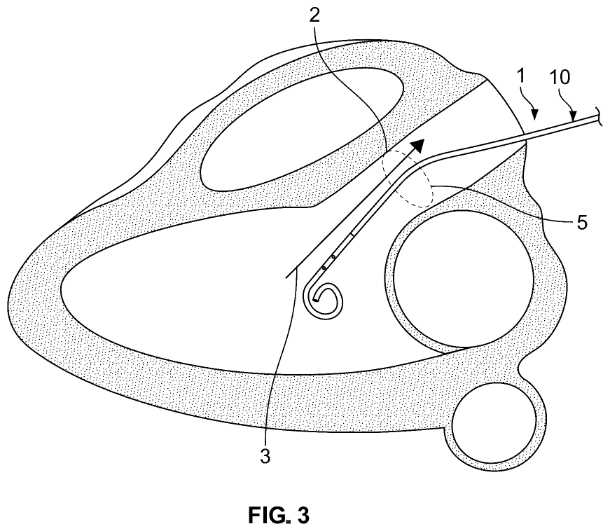

A method of measuring a cardiac output (“CO”) and/or a cardiac index (“CI”) with the cardiac output measuring system 4 is now described with reference to FIGS. 3-5. For ease of understanding, an exemplary method of measuring CO and/or CI with the cardiac output measuring system 4 is described below. Although the method is described in a specific order, the sequence of the steps of the method are merely exemplary; the sequence of the steps may be changed, additional steps may be added, and/or steps may be completed simultaneously.

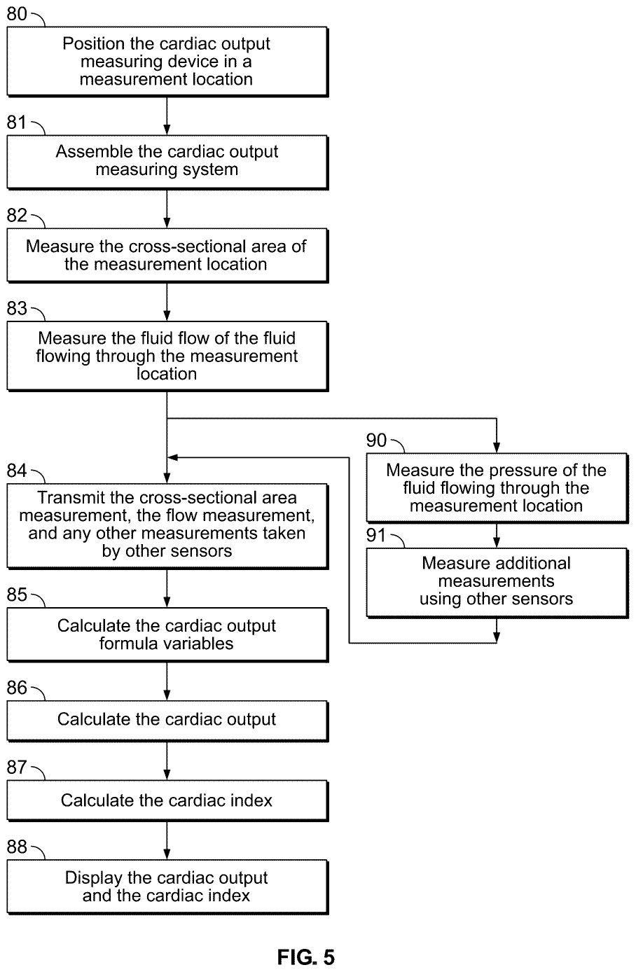

In the exemplary method of measuring CO and/or CI with the cardiac output measuring system 4, the cardiac output measuring device 1 is positioned in a measurement location 2 in a first step 80 as shown in FIG. 5. The measurement location 2 may be, for example, a left ventricular outflow tract “LVOT”, as shown schematically in FIG. 3. First, an incision is made on a body part of a patient to access a blood vessel. Second, the guidewire 18 is inserted through the inner lumen 16. The catheter 10 with the guidewire 18 is then inserted into the blood vessel. The guidewire 18 is used to thread the cardiac output measuring device 1, via the catheter 10, through the blood vessel and into the heart of the patient until it is positioned at a desired measurement location 2 shown in FIG. 3. In an embodiment, fluoroscopy is used to monitor the threading of the cardiac output measuring device 1 through the blood vessel and to ensure that the cardiac output measuring device 1 is placed in the desired measurement location 2. Once the cardiac output measuring device 1 is placed in the desired measurement location 2, the guidewire 18 can be removed from the inner lumen 16. The inner lumen 16 can then be used for various diagnostic procedures or therapeutic procedures such as injecting a dye, injecting an anesthetic, injecting a medicine, etc., into the patient at the measurement location 2.

The cardiac output measuring system 4 is then assembled by connecting the cardiac output measuring device 1, the connector 60, and the console 70 to one another in a second step 81, as shown in FIG. 5. First, the connector 60 is attached to the proximal end 12 of the catheter 10, with a terminal of the connector 60 in communication with the at least one electrical wire 20. The connector 60 is then attached to the console 70, for example by mating the connector 60 with a mating connector on the console 70. Once the connector 60 is attached to the console 70, an electrical connection 61 is formed between the cardiac output measuring device 1 and the console 70, as schematically shown in FIG. 4. The connector 60 places the elements of the cardiac output measuring device 1 in communication with the elements of the console 70, thereby facilitating the transfer of information from the cardiac output measuring device 1 to the console 70. In other embodiments, the step 81 may be performed before the step 80.

A cross-sectional area 5 of the measurement location 2 in the LVOT, as shown in FIG. 3, is then measured in a third step 82 shown in FIG. 5. The imaging sensor 30 provides 360° imaging, around the catheter 10, of the cross-sectional area 5 of the measurement location 2 to generate a cross-sectional area data 33. The imaging sensor 30 generates the cross-sectional area data 33 by using sound waves to create a two-dimensional echocardiogram image of the cross-sectional area 5. The sound waves are generated by the transducer 31 and bounced back towards the imaging sensor 30, where they are processed by the imaging integrated circuit 32. In an embodiment, the imaging integrated circuit 32 generates the cross-sectional area data 33 as a measurement of the cross-sectional area 5 through analysis of the data gathered by the transducer 31. In another embodiment, the imaging integrated circuit 32 generates an image of the measurement location 2 from the cross-sectional area data 33. The cross-sectional area data 33 is updated in real time; the cross-sectional area data 33 will change if the measurement location 2 has a dimensional change or a location of the measurement location 2 shifts to a different location with a different cross-sectional area 5.

A velocity of the fluid 3, i.e. blood, flowing through the measurement location 2, shown in FIG. 3, is then determined by using the flow sensor 40 in a fourth step 83, as shown in FIG. 5. The flow sensor 40 captures a flow measurement 41 of the fluid 3 by using sound waves. The sound waves released by the flow sensor 40 bounce off blood cells back towards the flow sensor 40 and are then measured for the flow measurement 41. The flow measurement 41 is a pulsed wave doppler that gathers the velocity of the fluid 3 over a period of time. The flow measurement 41 is updated in real time; the flow measurement 41 will change if the velocity of the fluid 3 increases or decreases.

The cross-sectional area data 33, the flow measurement 41, and any other measurements taken by the cardiac output measuring device 1 are then transmitted to the console 70 in a fifth step 84 as shown in FIG. 5. As shown in FIG. 4, the cross-sectional area data 33, the flow measurement 41, and any other measurements taken by the cardiac output measuring device 1 are transmitted through the at least one electrical wire 20 and the electrical connection 61 formed by the connector 60 to the console 70.

Data for the variables of a formula used for calculating CO are then determined by the console 70 in a sixth step 85, as shown in FIG. 5. An equation for calculating CO, stored on the memory 73 and executed by the processor 72, is:

CO=SV×HR (Equation 1)

In Equation 1, SV is stroke volume and HR is heart rate, the number of heart beats of the heart per minute. HR may be measured by a heart rate monitor connected to the patient and the console 70 and/or input into the console 70 based on the patient's measured HR using a device separate from the cardiac output measuring system 4.

SV is calculated according to the following equation:

SV=CX×DVTI (Equation 2)

In Equation 2, CX is a cross-sectional area of the LVOT and DVTI is a measurement of a distance the fluid 3 moves during a heartbeat. SV according to Equation 2 is thus the volume of the fluid 3 moved during a heartbeat, or an amount of fluid ejected from a left side of the heart per heartbeat. In an embodiment, SV is in units of mL, CX is in units of cm², and DVTI is in units of cm.

CO is then calculated by the console 70 in a seventh step 86 shown in FIG. 5.

The console 70 first determines CX by analyzing the cross-sectional area data 33. In an embodiment in which the cross-sectional area data 33 transmitted from the imaging integrated circuit 32 is a measurement of the cross-sectional area 5, with image analysis taking place at the imaging integrated circuit 32, the console uses the cross-sectional area data 33 directly as the value CX. In another embodiment in which the cross-sectional area data 33 transmitted from the imaging integrated circuit 32 is an image taken at the measurement location 2, the processor 72 analyzes the cross-sectional area data 33 and measures the cross-sectional area 5 from the image in the cross-sectional area data 33. For example, with a known scale of the image in the cross-sectional area data 33, the processor 72 executes an image analysis algorithm on the memory 73 to identify the boundary of the LVOT in the image and calculate an area of the image within the boundary.

Next, still in the calculation step 86, the console 70 determines DVTI from the flow measurement 41. The flow measurement 41 is representative of the velocity of the fluid 3 at the measurement location 2. The processor 72 determines a velocity time integral (“VTI”) from the flow measurement 41, executing an algorithm in the memory 73 to determine the change in velocities over time in the period of the flow measurement 41, calculating an area under the curve to determine how far the fluid 3 travels during the period of the flow measurement 41. This calculation of the distance that the fluid 3 traveled based on the flow measurement 41 is the DVTI.

The console 70 then, by execution of the processor 72, calculates the SV according to Equation 2 above, and calculates CO according to Equation 1 above. As described above, HR may be measured by a heart rate monitor connected to the patient and the console 70 and/or input into the console 70 based on other measurements of the patient's HR.

CI is then determined by the console 70 in an eighth step 87 as shown in FIG. 5. CI is CO normalized to the patient's body surface area (“BSA”). CI is calculated according to the following equation:

CI=CO/BSA (Equation 3)

BSA is determined by using a formula such as the Du Bois and Du Bois formula [BSA =0.007184×(Height in centimeters)\hat{ }0.725×(Weight in kilograms)\hat{ }0.425], the Mosteller formula, or the Haycock formula. Each of the formulas use the patient's height and weight as variables in calculating BSA. The height and weight of the patient or BSA of the patient is entered into the console 70. The processor 72 then either calculates BSA based on one of the aforementioned BSA formulas or inputs BSA into Equation 3. CI is then calculated by the processor 72 using Equation 3.

CO and the CI are displayed on the display 71 in a ninth step 88 as shown in FIG. 5. In an embodiment of the method, other information may be displayed on the display 71 such as HR, SV, DVTI, blood oxygenation, the cross-sectional area data 33 in the form of a two-dimensional echocardiogram image, the flow measurement 41 as a value or a two-dimensional DVTI image, and any other relevant patient physiological information.

In an embodiment of the method, after the fourth step 83, the pressure of the fluid 3 flowing through the measurement location 2 may be determined by the pressure sensor 50 in a step 90 as shown in FIG. 5. The pressure sensor 50 has a pressure sensitive element, such as a diaphragm. The diaphragm is deformed or deflected when pressure by the fluid 3 is exerted on the diaphragm, resulting in a voltage change. The pressure sensor 50 then calculates a pressure of the fluid 3 based on the deformation or deflection, which is recorded by the pressure sensor 50 as a pressure measurement 51. In an embodiment of the method, during the sixth step 85, along with calculating the CO formula variables the console 70, also calculates the pressure of the fluid 3 based on data transmitted to it from the pressure sensor 50, and can display the pressure in the step 88 described above.

In an embodiment of the method, additional measurements of temperature, oxygenation, or other measurements may be measured using a temperature sensor, an oxygenation sensor, or various other sensors of the cardiac output measuring device 1 in a step 91 shown in FIG. 5.

The cardiac output measuring system 4 and the cardiac output measuring device 1 according to the embodiments described herein generate a more accurate measurement of CO by directly measuring the actual cross-section area 5 of the measurement location 2 in the LVOT. This direct measurement results in a more accurate representation of SV, and correspondingly more accurate determinations of other values depending on the SV calculation like CO and CI, than assuming a circular cross-sectional shape. CO is calculated with all direct measurement, omitting assumed values that lead to inaccuracies. Further, positioning the flow sensor 40 adjacent to the imaging sensor 30 ensures the accuracy of the CO measurement, as both the velocity data used to calculate DVTI and the imaging data used to calculate CX are taken in the same location; even if the measurement location 2 has a dimensional change or the cardiac output measuring device 1 shifts, causing the sensors 30, 40 to measure a different measurement location 2, the sensors 30, 40 will shift together. Additionally, the cardiac output measuring device 1 requires a left heart catheterization without a right heart catheterization (i.e. a second procedure with a second access point and inherent risk) to obtain a measurement of CO.

What is claimed is:

1. A cardiac output measuring device, comprising:
a catheter having a distal end and a proximal end;
an imaging sensor positioned at the distal end and measuring a cross-sectional area of a measurement location; and
a flow sensor adjacent to the imaging sensor and measuring a velocity of a fluid passing through the measurement location.

2. The cardiac output measuring device of claim 1, wherein the imaging sensor is a B-mode ultrasound sensor.

3. The cardiac output measuring device of claim 2, wherein the imaging sensor has a transducer providing a 360° image around the catheter.

4. The cardiac output measuring device of claim 3, wherein the flow sensor is a doppler sensor.

5. The cardiac output measuring device of claim 4, wherein the flow sensor is parallel to a plane of the catheter and perpendicular to the transducer of the imaging sensor.

6. The cardiac output measuring device of claim 1, wherein the catheter is a pigtail catheter having a curved portion adjacent to the distal end.

7. The cardiac output measuring device of claim 6, wherein the catheter has an end hole on the curved portion and a side hole at the distal end.

8. The cardiac output measuring device of claim 7, further comprising a pressure sensor in communication with the end hole or the side hole.

9. A method of measuring cardiac output comprising:
positioning a catheter in a heart;
measuring a cross-sectional area of a measurement location in the heart to capture a cross-sectional area measurement;
measuring a velocity of a fluid passing through the measurement location to capture a flow measurement; and
calculating a cardiac output based on the cross-sectional area measurement and the flow measurement.

10. The method of claim 9, wherein the catheter is positioned in a left ventricular outflow tract of a left ventricle of the heart, the measurement location is a cross-section of the left ventricular outflow tract.

11. The method of claim 9, wherein the cardiac output (CO) is calculated according to:
CO=SV×HR,
where SV is a stroke volume calculated from the cross-sectional area measurement and the flow measurement, and HR is a heart rate.

12. The method of claim 11, wherein the stroke volume SV is calculated according to:
SV=CX×DVTI,
where CX is the cross-sectional area of the measurement location determined by the cross-sectional area measurement and DVTI is a doppler velocity time integral determined by analyzing the flow measurement.

13. The method of claim 12, further comprising calculating a cardiac index (CI) according to
CI=CO/BSA,
where BSA is a body surface area that depends on a height and a weight of a patient.

14. The method of claim 9, further comprising measuring a pressure of a fluid passing through the measurement location.

15. A cardiac output measuring system comprising:
a cardiac output measuring device including a catheter having a distal end and a proximal end, an imaging sensor positioned at the distal end and measuring a cross-sectional area of a measurement location to capture a cross-sectional area measurement, and a flow sensor adjacent to the imaging sensor and measuring a velocity of a fluid passing through the measurement location to capture a flow measurement; and
a console connected to the cardiac output measuring device and receiving the cross-sectional area measurement and the flow measurement from the cardiac output measuring device.

16. The cardiac output measuring system of claim 15, wherein the imaging sensor is a B-mode ultrasound sensor and the console calculates the cross-sectional area of the measurement location by analyzing an image obtained by the imaging sensor.

17. The cardiac output measuring system of claim 16, wherein the flow sensor is a doppler sensor and the console calculates a doppler velocity time integral from the flow measurement.

18. The cardiac output measuring system of claim 17, wherein the console calculates a stroke volume based on the cross-sectional area of the measurement location and the doppler velocity time integral from the flow measurement.

19. The cardiac output measuring system of claim 18, wherein the console calculates a cardiac output based on the stroke volume and a heart rate of a patient.

20. The cardiac output measuring system of claim 15, further comprising a connector attached at the proximal end of the catheter, the connector connects to the console and forms an electrical connection between the cardiac output measuring device and the console.
